(19) United States
(12) Patent Application Publication
(10) Pub. No.: US 2025/0228959 A1
(43) Pub. Date: Jul. 17, 2025

(54) TRANSFERRIN-BASED LYSOSOME TARGETING DEGRADERS

(71) Applicant: Wisconsin Alumni Research Foundation, Madison, WI (US)

(72) Inventors: Weiping Tang, Middleton, WI (US); Yaxian Zhou, Madison, WI (US)

(73) Assignee: Wisconsin Alumni Research Foundation, Madison, WI (US)

(21) Appl. No.: 18/848,718

(22) PCT Filed: Mar. 22, 2023
(86) PCT No.: PCT/US2023/015943

§ 371 Date: Sep. 19, 2024

Related U.S. Application Data
(60) Provisional application No. 63/322,433, filed on Mar. 22, 2022.
(60) Provisional application No. 63/407,747, filed on Sep. 19, 2022.

(51) Int. Cl. A61K 47/68

(52) U.S. Cl. CPC A61K 47/6811

(57) ABSTRACT

Provided herein are bifunctional lysosomal targeting degraders including a ligand configured to bind to transferrin receptor as a shuttle molecule for lysosome degradation, and a protein-binding moiety configured to bind a membrane or extracellular protein of interest, wherein the bifunctional lysosome targeting degraders can be recycled back out of cells and be catalytic. The bifunctional degraders find use, e.g., for selectively targeted degradation of membrane or extracellular proteins via the endosomal/lysosomal pathway. Also provided herein are compositions comprising the bifunctional degraders, as well as methods of using the bifunctional degraders.

### CROSS-REFERENCE TO RELATED APPLICATIONS

10 Priority is hereby claimed to provisional application Ser. No. 63/407,747, filed Sep. 19, 2022, and to provisional application Ser. No. 63/322,433, filed Mar. 22, 2022, the content of which are incorporated herein by reference.

### FEDERAL FUNDING STATEMENT

This invention was made with government support under GM120357 awarded by the National Institutes of Health. The government has certain rights in the invention.

### BACKGROUND

Targeted Protein Degradation (TPD) is emerging as an exciting therapeutic option to confront diseases involving aberrantly expressed or mutated disease-causing proteins by engaging our body's natural protein disposal systems. TPD by chimeric molecules is a novel therapeutic modality (Deshaies, 2020, Nature.580:329-338). These chimeras are heterobifunctional molecules with one end binding to the protein of interest (POI) and the other end directing the resulting complex towards a certain degradation pathway. PROteolysis TArgeting Chimera (PROTAC) has received the most attention to date. (See Sakamoto et al., 2001, Proc. Natl. Acad. Sci. 98:8554-8559; Luh et al., 2020, Angew. Chem. Int. Ed. 59:15448-15466; Wu et al., 2020, Nat. Struct. Mol. Biol. 27:605-614.) PROTACs contain an E3 ligase ligand to route the targeted protein to the proteasome for degradation. (Lai and Crews, 2017, Nat. Rev. Drug Discovery. 16:101-114; Salami and Crews, 2017, Science. 355:1163-1167; Cromm and Crews, 2017, Cell Chem. Biol. 24:1181-1190; Toure and Crews, 2016, Angew. Chem. Int. Ed. 55:1966-1973.) However, PROTACs are only capable of depleting intracellular proteins. There are many disease targets that are membrane or extracellular proteins.

To broaden the scope of targets, researchers have reported a way of tagging extracellular protein targets with a ligand for membrane receptors involved in active transport of molecules into the cell. The tagged protein is naturally shuttled to the lysosome in the cell where it is degraded. The bifunctional lysosome targeting degraders are generally created by conjugating ligands of the lysosome targeting receptors (LTRs) on the cell surface with ligands that can bind to the extracellular protein target. This process is shown schematically in FIG. 1. The LTRs employed in the past studies are carbohydrate binding proteins including cation-independent mannose 6-phosphate receptor (CIM6PR or insulin-like growth factor-II receptor) (Banik et al., 2020, Nature. 584:291-297) and asialoglycoprotein receptor (ASGPR) (Zhou et al., 2021, ACS Cent. Sci. 7:499-506; Ahn et al., 2021, Nat. Chem. Biol. 17:937-946; Caianiello et al., 2021, Nat. Chem. Biol. 17:947-953). The receptor-ligand interaction triggers the internalization of the extracellular proteins through receptor-mediated endocytosis, further inducing the degradation of the targets in the lysosome.

One type of bifunctional lysosome targeting degraders were developed by conjugating the ligand of the CIM6PR on the cell surface with a molecule that binds to the extracellular protein target (Banik et al., 2020, Nature. 584:291-297). This type of bifunctional lysosome-targeting degraders that recruit CIM6PR was also termed LYsosome TArgeting Chimeras (LYTACs). CIM6PR is expressed ubiquitously in most cell types. The receptor-ligand interaction triggers the internalization of the extracellular proteins through receptor-mediated endocytosis, further inducing the degradation of the targets in the lysosome. CIM6PR is a transmembrane receptor that transports proteins bearing N-glycans capped with mannose 6-phosphate (M6P) residues to lysosomes (Ghosh et al., 2003, Nat. Rev. Mol. Cell Biol. 4:202-213; Coutinho et al., 2012, Mol. Genet. Metab. 105:542-550). Early studies showed that albumin modified with M6P increased the cellular uptake (Beljaars et al., 1999, Hepatology. 29:1486-1493). Subsequently, CIM6PR was used to deliver therapeutic drugs conjugated with M6P derivatives for lysosomal enzyme replacement therapy and cancer treatment (Ghosh et al., 2003, Nat. Rev. Mol. Cell Biol. 4:202-213; Gary-Bobo et al., 2007, Curr. Med. Chem. 14:2945-2953). Various molecules, such as peptides, proteins or liposome, were covalently linked to the M6P or its analogues to achieve targeted drug delivery (Hoogendoorn et al., 2014, Angew. Chem. Int. Ed. 53:10975-10978; Crucianelli et al., 2014, RSC Adv. 4:58204-58207; Das et al., 2016, Acs Macro Letters. 5:809-813; Agarwal et al., 2016, Chem. Commun. 52:327-330; Hyun et al., 2018, Cell Chem. Biol. 25:1255-1267). To extend the usage of CIM6PR/M6P system to targeted protein degradation, LYTAC was constructed by conjugating a mixture of polyglycopeptides containing 20-40 units of M6P analogues to the antibody of POI. Different from drug delivery process, which involves the internalization of a covalent linked M6P-protein target, LYTAC allows the trafficking of a complex formed by the non-covalent interaction between the protein target and LYTAC. It was shown that LYTAC could successfully degrade both secreted and membrane proteins in the lysosome through CIM6PR (Banik et al., 2020, Nature. 584:291-297). However, the challenge associated with the synthesis and attachment of a heterogenous mixture of polymeric glycopeptides with 20-40 units of M6P analogues to antibodies employed in the LYTAC system limited its utility in drug development. In addition, since CIM6PR is ubiquitously expressed in most cell types, the POI is delivered to all cell types non-selectively. There remains an unmet need for selective means of degrading extracellular proteins.

### SUMMARY

Disclosed herein is a bifunctional lysosomal targeting degrader. The degrader comprises a ligand configured to bind to a transferrin receptor as a shuttle molecule for lysosome degradation, operationally linked to a protein-binding moiety configured to bind a membrane or extracellular protein. The bifunctional lysosome targeting degrader itself can be recycled out of cells. It may also have catalytic activity. As used herein, “operationally linked” means that the transferrin receptor ligand may be linked directly or indirectly to the protein binding moiety. Thus a direct link would comprise a direct chemical bond between the two moieties (without any intervening atoms) or there may be a linking moiety (“linker”) of any description between the transferrin receptor and the protein binding moiety.

In one version of the bifunctional degrader, the ligand that binds to the transferrin receptor is transferrin itself. In other versions, the ligand that binds to the transferrin receptor is an antibody that binds to the transferrin receptor.

The protein-binding moiety of the bifunctional degrader may be configured to bind a membrane protein. By way of example, and not limitation, the membrane protein bound by the protein-binding moiety may be a membrane receptor, an immune inhibitory receptor, a ligand of an immune inhibitory receptor, an immune checkpoint molecule, and the like.

In still other versions of the bifunctional degrader, the protein-binding moiety of the bifunctional degrader binds an extracellular protein. The extracellular protein may be, for example, a ligand for a membrane receptor, an auto-antibody, a secreted protein, or a mutated protein.

The protein-binding moiety of the bifunctional degrader may be any type of moiety configured to bind to a membrane or extracellular protein to be targeted for degradation via the endosomal/lysosomal pathway. For example, the protein-binding moiety may be selected from the group consisting of polypeptides, ligands, aptamers, nanoparticles, and small-molecule binders.

In some embodiments, the bifunctional degrader disclosed herein includes a ligand of transferrin receptor conjugated to the protein-binding moiety. In other embodiments, one or more linkers may be employed to facilitate conjugation of the ligand of transferrin receptor to the protein-binding moiety.

When the ligand of transferrin receptor and the protein-binding moiety are both polypeptides, the bifunctional degrader disclosed herein comprises a fusion protein containing the transferrin receptor ligand fused to the protein-binding moiety. The bifunctional degrader may optionally further comprise a spacer between the transferrin receptor ligand and the protein-binding moiety. When the bifunctional degrader comprises a fusion protein, it may be expressed in cells by introducing to the cells an expression vector that contains nucleic acids encoding the bifunctional degrader.

By binding to transferrin receptor, the bifunctional lysosomal targeting degrader disclosed herein selectively targets cells that express transferrin receptors to degrade membrane or extracellular proteins of interest. In some embodiments, the cells that express transferrin receptors are cancer cells.

Also provided herein is a pharmaceutical composition that includes any of the bifunctional lysosomal targeting degraders of the present disclosure. The pharmaceutical compositions may optionally further comprise a pharmaceutically acceptable carrier.

The disclosure further encompasses a method of degrading a membrane or extracellular protein. The method comprises contacting the membrane or extracellular protein with any of the bifunctional lysosomal targeting degraders of the present disclosure, wherein the bifunctional lysosomal targeting degrader shuttles the membrane or extracellular protein to lysosome for degradation and the bifunctional lysosomal targeting degrader can be recycled back out of cells and be catalytic.

Also disclosed herein is a method that includes administering to an individual in need thereof a therapeutically effective amount of any of the pharmaceutical compositions of the present disclosure. The individual may be a human. In some embodiments, the individual has a cancer and the pharmaceutical compositions are administered to the subject in an amount effective to inhibit growth/progression of the cancerous cells.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1 is a schematic diagram illustrating chimeric degrader-induced degradation of extracellular protein of interest (POI) through lysosome targeting receptor (LTR).

FIG. 2 is a schematic diagram illustrating degradation of target protein by transferrin-based catalytic cancer cell-specific LYTACs (transferrin-LYTAC).

FIG. 3 is a schematic diagram illustrating degradation of target protein by transfecting cells with a plasmid expressing transferrin-based catalytic LYTACs (transferrin-LYTAC).

FIG. 4 presents protein immunoblots showing uptake of anti-biotin-647 (IgG-647) in MCF-7 (upper panel) and Huh7 (middle and lower panels) cells treated with biotin-labelled transferrin (Tf-Biotin) in regular media with serum (25 nM of Tf-biotin and 50 nM of IgG-647).

FIG. 5 presents protein immunoblots showing uptake of anti-biotin-647 (IgG-647) in Huh7 cells treated with biotin-labelled transferrin in media without serum.

FIG. 6 presents protein immunoblots illustrating the chase phase of the pause-chase experiments testing if the biotin-labelled transferrin (Tf-biotin) can travel through the membrane in both directions.

FIG. 7 presents protein immunoblots showing uptake of anti-FLAG-647 in cells transfected with plasmid expressing transferrin with a FLAG-tag (“Tf-FLAG”). Upper panel shows 24 h of uptake and lower panel shows 36 h of uptake.

FIG. 8 presents protein immunoblots showing uptake of anti-biotin-647 in MCF-7 and Huh7 cells treated with peptides P7, P9 and P12 attached to the anti-biotin-647 antibodies for 6 h.

FIG. 9 presents protein immunoblots showing degradation of EGFR in A549, Huh7, and MCF-7 cells treated with cetuximab (Ctx) and peptides P7, P9 and P12 attached to Ctx with PEG3 as the linker for 48 h. Actin is used as loading control. “−” represents negative controls. Ctx labeled folate (Ctx-FA) is used as positive controls.

FIG. 10 presents protein immunoblots showing degradation of EGFR in HepG2, MCF7, and Hela cells treated with Ctx and peptides P7, P9 and P12 attached to Ctx with PEG3 or PEG 12 as the linker. “−” represents negative controls. Ctx-FA is used as positive controls. The effect is also compared to the Ctx attached to cRGD with PEG3 or PEG12 as the linker.

### DETAILED DESCRIPTION

Provided herein are bifunctional lysosome targeting degraders that comprise a ligand that is configured to bind to transferrin receptor as a shuttle molecule for lysosome degradation, and a protein-binding moiety that is configured to bind a membrane or extracellular protein of interest. The bifunctional lysosome targeting degraders can be recycled back out of cells and be catalytic. The bifunctional degrader disclosed herein finds use, for example, for selectively targeted degradation of membrane or extracellular proteins via the endosomal/lysosomal pathway. In one aspect, the bifunctional degraders induce degradation of oncogenic proteins specifically in cancer cells via the transferrin receptor, which is overexpressed in many cancer cells. Also provided herein are compositions comprising the bifunctional degraders, as well as methods of using the bifunctional degraders to inhibit the growth of neoplastic cells.

It is to be understood that the bifunctional degraders, compositions, and methods disclosed herein are not limited to particular embodiments described, and as such may vary. It is also to be understood that the terminology used herein is for the purpose of describing specific embodiments only and is not intended to be limiting.

It is noted that, as used herein and in the appended claims, the singular forms “a”, “an”, and “the” include plural referents unless the context clearly dictates otherwise. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

It is appreciated that certain features of the bifunctional degraders, compositions, and methods, which are (for purposes of clarity) described in the context of separate embodiments, may also be provided in combination in a single embodiment. Conversely, various features of the bifunctional degraders, compositions, and methods, which are, for brevity, described in the context of a single embodiment, may also be provided separately or in any suitable sub- combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present bifunctional degraders, compositions, and methods and are disclosed herein just as if each and every such sub-combination was individually and explicitly disclosed herein. It is also noted that the bifunctional degraders, compositions, and methods provided herein suitably may be practiced in the absence of any element which is not specifically disclosed herein.

Bifunctional lysosome targeting degraders:

Provided herein are bifunctional lysosome targeting degraders that include a ligand configured to bind to transferrin receptor as a shuttle molecule for lysosome degradation, and a protein-binding moiety that configured to bind a membrane or extracellular protein of interest, wherein the bifunctional lysosome targeting degraders can be recycled back out of cells and be catalytic.

In some embodiments, the ligand that binds to transferrin receptor is transferrin (Tf). Tf is an iron-binding glycoprotein that facilitates iron uptake in cells. It contains binding sites for two Fe³⁺ions (Hall et al., 2002, Acta Crystallogr. D ACTA CRYSTALLOGR D. 58:70-80). The trafficking pathway of Tf has been well documented (Dautryvarsat et al., 1983, Proc. Natl. Acad. Sci. U.S.A. 80:2258-2262; Yamashiro et al., 1984, Cell. 37:789-800; Qian et al., 2002, Pharmacol Rev. 54:561-587; Mayle et al., 2012, Biochim. Biophys. Acta. 1820:264-281). Apo-Tf (iron-free Tf) binds to one or two iron atoms outside of the cells and enters the cell by binding to the cell-surface Tf receptor through clathrin-mediated endocytosis. After releasing iron in early endosomes, apo-Tf together with its receptor are directed to recycling endosome to be taken back to the cell surface. Finally, apo-Tf dissociates from its receptor and enters the solution to take another molecule of iron. The fast kinetics for the recycling of Tf makes it ideal carrier for the catalytic lysosome targeting degraders. It has been shown that the T_{1/2}s are 3.5 min and 5 min for the endocytosis of surface-bond Tf and secretion of the apo-Tf, respectively, in HepG2 cells (Ciechanover et al., 1983, J. Biol. Chem. 258:9681-9689).

The ligand that binds to transferrin receptor may comprise a transferrin receptor antibody.

The ligand that specifically binds to transferrin receptors may comprise peptide binders of the transferrin receptor, including, but not limited to P7 (HAIYPRH; also known as T7) (SEQ ID NO: 1), P9 (GHKAKGPRK; also known as B6) (SEQ ID NO:2), and P12 (THRPPMWSPVWP; also known as T12 or THR) (SEQ ID NO:3). The three peptides have been used for drug delivery (Xia et al., 2000, J. Virol. 74, 11359; Lee et al., 2001, Eur. J. Biochem. 268, 2004; Mojarad-Jabali et al., 2022, Int. J. Pharm. 613, 121395). They showed blood-brain barrier (BBB) penetration capacity and brain distribution.

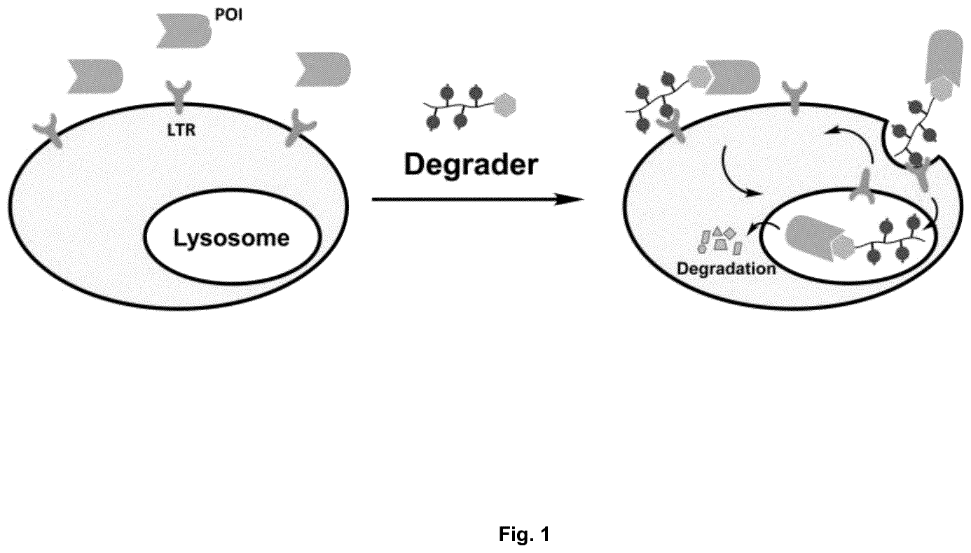

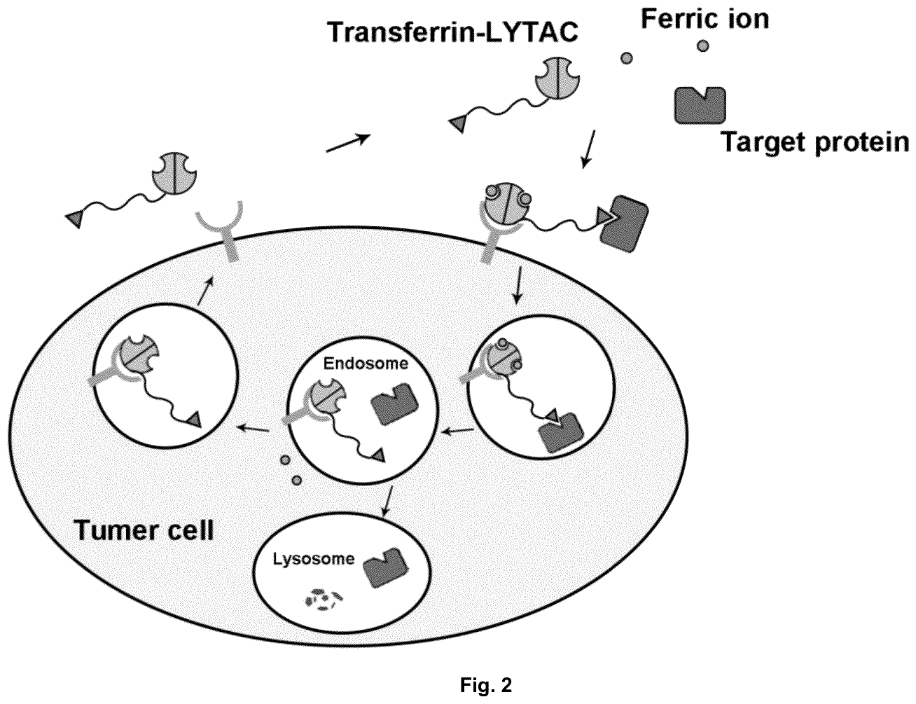

As disclosed herein (FIGS. 1 and 2), the ligand of the transferrin receptor (e.g., transferrin) is attached to a binder of an extracellular protein of interest creating a transferrin-LYTAC. Transferrin acts as a shuttle to bring the protein of interest into the cell. Once inside the cell, transferrin enters an endosome where it deposits the protein of interest payload analogous to how it deposits iron in the endosome. Transferrin leaves the endosome and encounters its receptor. The transferrin receptor transports the transferrin-protein binding shuttle back outside the cell where it can bind another protein and shuttle it inside the cell. The released target protein is degraded in the lysosome.

Transferrin receptor (TfR) is a cell-membrane-associated glycoprotein involved in the cellular uptake of iron and the regulation of cell growth (Neckers and Trepel, 1986, Cancer Invest. 4:461-470). Transferrin receptor 1 (TfR1, also known as CD71), is ubiquitously expressed at low levels in most normal human tissues. A second member of the TfR family is TfR2, a protein that is homologous to TfRI but whose expression is largely restricted to hepatocytes (Daniels et al., 2006, Clin. Immunol. 121:144-158). Serving as the main port of entry for iron bound Tf into cells, TfR1 is a type-II receptor that resides on the cell membrane and cycles into acidic endosomes into the cell in a clathrin/dynamin dependent manner (Daniels et al., 2006, Clin. Immunol. 121:144-158; Cheng et al., 2004, Cell. 116:565-576; Lebron et al., 1998, Cell. 93:111-123). Iron is delivered into the cell and TfR1 is recycled back to the cell surface (Daniels et al., 2006, Clin. Immunol. 121:144-158; Hemadi et al., 2004, Biochemistry. 43:1736-1745; Ciechanover et al., 1983, J. Cell. Biochem. 23:107-130). Despite its ubiquitous expression, TfR1 is expressed on malignant cells at levels several fold higher than those on normal cells and its expression can be correlated with tumor stage or cancer progression (Yang et al., 2001, Anticancer Res. 21:541-549; Prior et al., 1990, Virchows Arch. A Pathol. Anat. Histopathol. 416:491-496; Kondo et al., 1990, Chest. 97:1367-1371). Due to the overexpression of transferrin receptor on cancer cells, the bifunctional degraders disclosed herein impart selectivity to cancer cells to degrade oncogenic membrane or extracellular proteins.

As disclosed herein, the bifunctional lysosome targeting degraders include a protein-binding moiety that is configured to bind a membrane or extracellular protein of interest. In some embodiments, the protein-binding moiety binds a membrane protein.

The membrane protein is typically (but not necessarily) a membrane receptor. Membrane receptors of interest include, but are not limited to, stem cell receptors, immune cell receptors, growth factor receptors, cytokine receptors, hormone receptors, receptor tyrosine kinases, a receptor in the epidermal growth factor receptor (EGFR) family (e.g., HER2 (human epidermal growth factor receptor 2), etc.), a receptor in the fibroblast growth factor receptor (FGFR) family, a receptor in the vascular endothelial growth factor receptor (VEGFR) family, a receptor in the platelet derived growth factor receptor (PDGFR) family, a receptor in the rearranged during transfection (RET) receptor family, a receptor in the Eph receptor family, a receptor in the discoidin domain receptor (DDR) family, and a mucin protein (e.g., MUC1).

The membrane protein may be (for example) an immune inhibitory receptor. As used herein, an “immune inhibitory receptor” is a receptor present on an immune cell that negatively regulates an immune response. Examples of inhibitory immune receptors include immune inhibitory receptors of the Ig superfamily, including but not limited to: CD200R, CD300a (IRp60; mouse MAIR-I), CD300f (IREM-1), CEACAMI (CD66a), FcyRIIb, ILT-2 (LIR-1; LILRB1; CD85j), ILT-3 (LIR-5; CD85k; LILRB4), ILT-4 (LIR-2; LILRB2), ILT-5 (LIR-3; LILRB3; mouse PIR-B); LAIR-1, PECAM-1 (CD31), PILR-α (FDF03), SIRL-1, and SIRP-α. Further examples of immune inhibitory receptors include sialic acid-binding Ig-like lectin (Siglec) receptors, e.g., Siglec 7, Siglec9, and/or the like. Additional examples of immune inhibitory receptors include C-type lectins, including but not limited to: CLEC4A (DCIR), Ly49Q and MICL. Details regarding immune inhibitory receptors may be found, e.g., in Steevels et al., 2011, Eur. J. Immunol. 4:575-587.

The membrane protein can be a ligand of an immune inhibitory receptor.

On other versions of the bifunctional degraders, the membrane protein may be an immune checkpoint molecule including immune checkpoint proteins and ligands. Non-limiting examples of immune checkpoint molecules include PD-1, PD-L1, CTLA4, TIM3, LAG3, TIGIT, and members of the B7 family.

As disclosed herein, the bifunctional lysosome targeting degraders include a protein-binding moiety that is configured to bind a membrane protein or extracellular protein of interest. In some embodiments, the protein-binding moiety binds an extracellular protein.

Thus, for example, the extracellular protein can be a ligand for a membrane receptor. Membrane receptor ligands of interest include, but are not limited to, growth factors (e.g., epidermal growth factor (EGF), vascular endothelial growth factor (VEGF), and the like), cytokines (e.g., an interleukin, an interferon, a tumor necrosis factor (TNF), a transforming growth factor β (TGF-β), including any particular subtypes of such cytokines), hormones, and the like.

The extracellular protein target can also be an antibody, such as an antibody that binds a membrane protein or a different extracellular protein. The antibody can be an autoantibody. By “autoantibody” is meant an antibody produced by the immune system that is directed against one or more of the individual's own proteins. Cancer cells can induce an immunological response resulting in the production of tumor-associated autoantibodies. Non-limiting examples of autoantibodies include rheumatoid factor (RF), antinuclear antibody (ANA), antineutrophil cytoplasmic antibodies (ANCA), anti-double stranded DNA (anti-dsDNA), anticentromere antibodies (ACA), anticyclic citrullinated peptide antibodies (anti-CCP), extractable nuclear antigen antibodies (ENA), anticardiolipin antibodies, beta-2 glycoprotein 1 antibodies, antiphospholipid antibodies (APA), lupus anticoagulants (LA), anti-tissue transglutaminase (anti-tTG), anti-gliadin antibodies (AGA), intrinsic factor antibodies, parietal cell antibodies, thyroid antibodies, smooth muscle antibodies (SMA), antimitochondrial antibodies (AMA), anti-glomerular basement membrane (GBM), acetylcholine receptor (AChR) antibodies, etc.

The extracellular protein may also be a secreted protein, including secreted growth factors and factors, extracellular matrix-degrading proteinases, cell motility immunoregulatory cytokines or other bioactive molecules.

The extracellular protein may also be a mutated protein.

In a preferred version, the protein-binding moiety of the bifunctional degrader binds a membrane or extracellular protein on a cancer cell or produced by a cancer cell. By “cancer cell” is meant a cell exhibiting a neoplastic cellular phenotype, which may be characterized by one or more of, for example, abnormal cell growth, abnormal cellular proliferation, loss of density-dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like.

The protein-binding moiety of the bifunctional degrader may be any type of moiety capable of binding to the membrane or extracellular protein to be targeted for degradation via the endosomal/lysosomal pathway. In certain aspects, the protein-binding moiety is selected from a polypeptide, a ligand (e.g., a ligand for a membrane receptor, where the membrane receptor is targeted for degradation), an aptamer, a nanoparticle, and a small molecule. In certain embodiments, the protein-binding moiety is a small molecule. By “small molecule” is meant a compound having a molecular weight of about 1000 atomic mass units (amu) or less. In some embodiments, the small molecule is about 750 amu or less, about 500 amu or less, about 400 amu or less, about 300 amu or less, or about 200 amu or less. In certain embodiments, the small molecule binds to the target membrane or extracellular protein and allows the protein to dissociate from the small molecule in an endosome without coming out of the cells together with the bifunctional degrader.

The protein-binding moiety may also be a polypeptide, such as an antibody. The terms “antibody” and “immunoglobulin” include antibodies or immunoglobulins of any isotype (e.g., IgG (e.g., 1gG1, 1gG2, 1gG3 or 1gG4), IgE, IgD, IgA, IgM, etc.); whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to the membrane or extracellular protein, including, but not limited to, Fv, single chain Fv (scFv), Fab, F (ab′)₂, Fab′, (scFv′)₂, diabodies, and nanobodies; chimeric antibodies; monoclonal antibodies; fully human antibodies; humanized antibodies (e.g., humanized whole antibodies, humanized antibody fragments, etc.); and fusion proteins including an antigen-binding portion of an antibody and a non-antibody protein or fragment thereof. The antibodies may be detectably labeled, e.g., with an in vivo imaging agent, or the like. In certain embodiments, the antibody binds to a cancer antigen. In certain embodiments, the antibody binds to an intact complement or a fragment thereof. In some embodiments, the antibody binds to one or more immunodominant epitope(s) within intact complement or a fragment thereof.

The bifunctional lysosome targeting degraders disclosed herein may be in any suitable format. In some embodiments, the bifunctional degrader is a conjugate. Accordingly, in some embodiments, the bifunctional degrader disclosed herein includes the ligand of transferrin receptor conjugated to the protein-binding moiety (e.g., a small molecule). In certain embodiments, one or more linkers may be employed to facilitate conjugation of transferrin to the protein-binding moiety. Non-limiting examples of such linkers include ester linkers (e.g., N-hydroxysuccinimide (NHS) ester, sulfo-NHS ester or PFP ester or thioester), amide linkers, imine linkers, maleimide or maleimide-based linkers; valine-citrulline linkers; hydrazone linkers; N-succinimidyl-4-(2-pyridyldithio) butyrate (SPDB) linkers; Succinimidyl-4-(A/-maleimidomethyl)cyclohexane-1-carboxylate (SMCC) linkers; vinylsulfone-based linkers; linkers that include polyethylene glycol (PEG), such as, but not limited to tetraethylene glycol; linkers that include propanoic acid; linkers that include caproleic acid, and linkers including any combination thereof.

In certain aspects, the linker comprises at least one cleavable linking group. In certain embodiments, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. In certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry and Stump, 2010, Bioconjugate Chem. 21:5-13.

In certain embodiments, the bifunctional lysosome targeting degrader conjugates can be formed by covalently linking the ligand of transferrin receptor to the protein-binding moiety (e.g., a small molecule), either directly or through one or more linker molecules, or through one or more functional groups to form a covalent conjugate.

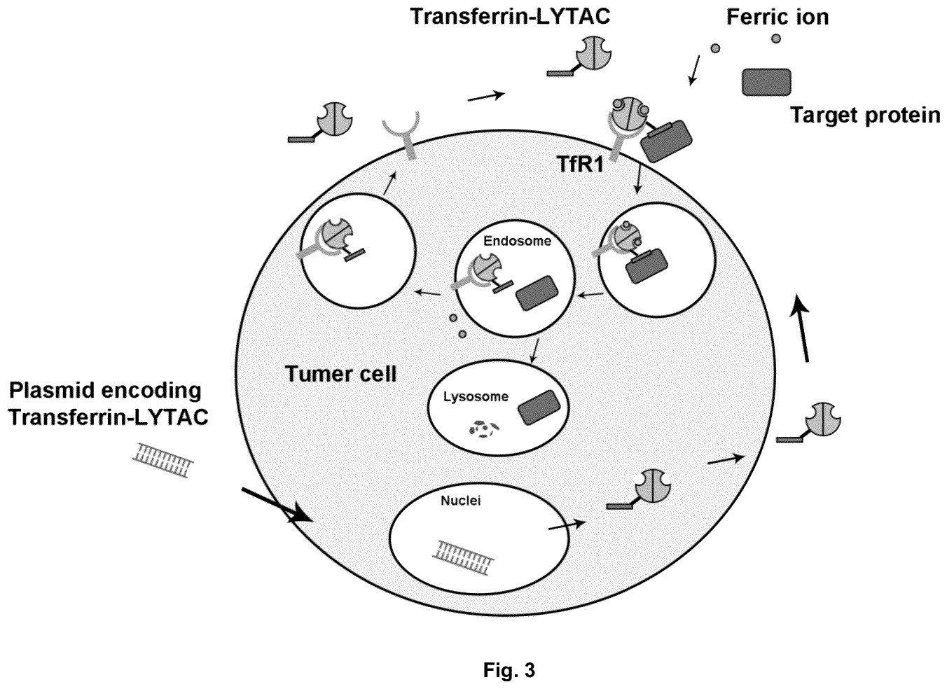

In some embodiments, when the ligand of transferrin receptor and the protein-binding moiety are both polypeptides, the bifunctional degrader is a fusion protein comprising the ligand of transferrin receptor fused to the protein-binding moiety. The ligand of transferrin receptor may be fused directly to the protein-binding moiety. In other aspects, the ligand of transferrin receptor may be fused indirectly to the protein-binding moiety, e.g., where a spacer is disposed between the ligand of transferrin receptor and the protein-binding moiety. In some embodiments, nucleic acids that encode the bifunctional degrader are present on an expression vector (e.g., plasmids). The bifunctional degrader is expressed in cells to degrade target proteins by introducing the expression vector into the cells (e.g., by transfection; see FIG. 3).

In certain aspects, the bifunctional degrader enhances degradation of the membrane or extracellular protein relative to degradation of the membrane or extracellular protein in the presence of the protein-binding moiety alone. According to some embodiments, the bifunctional degrader enhances degradation of the membrane or extracellular protein relative to degradation of the membrane or extracellular protein in the presence of the ligand of transferrin receptor or the protein-binding moiety alone. By “enhances degradation” in this context means the membrane or extracellular protein is degraded in the presence of the bifunctional degrader and is not degraded in the presence of the protein-binding moiety alone, or the presence of the ligand of transferrin receptor or the protein-binding moiety alone, under the same conditions; or the membrane or extracellular protein is degraded in the presence of the bifunctional degrader to a greater extent than the membrane or extracellular protein is degraded in the presence of the protein-binding moiety alone, or the presence of the ligand of transferrin receptor or the protein-binding moiety alone, under the same conditions. When the membrane or extracellular protein is degraded in the presence of the bifunctional degrader to a greater extent than the membrane or extracellular protein is degraded in the presence of the protein-binding moiety alone, or the presence of the ligand of transferrin receptor or the protein-binding moiety alone under the same conditions, the degradation may be 1.2 fold or greater, 1.4 fold or greater, 1.6 fold or greater, 1.8 fold or greater, 2 fold or greater, 2.5 fold or greater, 3 fold or greater, 3.5 fold or greater, 4 fold or greater, 4.5 fold or greater, 5 fold or greater, 5.5 fold or greater, 6 fold or greater, 6.5 fold or greater, 7 fold or greater, 7.5 fold or greater, 8 fold or greater, 8.5 fold or greater, 9 fold or greater, 9.5 fold or greater, or 10 fold or greater in the presence of the bifunctional degrader.

### Compositions:

Disclosed herein are compositions that include any of the bifunctional lysosomal targeting degraders in the present disclosure.

In certain aspects, the compositions include a bifunctional degrader of the present disclosure present in a liquid medium. The liquid medium may be an aqueous liquid medium, such as water, a buffered solution, and the like. One or more additives such as a salt (e.g., NaCl, MgCl₂, KCI, MgSO₄), a buffering agent (a Tris buffer, N-(2-Hydroxyethyl) piperazine-N′-(2-ethanesulfonic acid) (HEPES), 2-(N-Morpholino) ethanesulfonic acid (MES), 2-(N-Morpholino)ethanesulfonic acid sodium salt (MES), 3-(N-Morpholino)propanesulfonic acid (MOPS), N-tris [Hydroxymethyl] methyl-3-aminopropanesulfonic acid (TAPS), etc.), a protease inhibitor, glycerol, and the like may be present in such compositions.

Also disclosed herein are pharmaceutical compositions that include any of the bifunctional lysosomal targeting degraders of the present disclosure, and a pharmaceutically acceptable carrier. The pharmaceutical compositions generally include a therapeutically effective amount of the bifunctional degrader. By “therapeutically effective amount” is meant a dosage sufficient to produce a desired result, e.g., an amount sufficient to effect beneficial or desired therapeutic (including preventative) results, such as a reduction in cellular proliferation in an individual having a cell proliferative disorder (e.g., cancer) associated with the membrane or extracellular protein to which the protein-binding moiety of the bifunctional degrader binds, etc. An effective amount may be administered in one or more administrations.

A bifunctional degrader of the present disclosure can be incorporated into a variety of formulations for therapeutic administration. More particularly, the bifunctional degrader can be formulated into pharmaceutical compositions by combination with appropriate pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the bifunctional degraders of the present disclosure suitable for administration to an individual (e.g., suitable for human administration) are generally sterile and may further be free of detectable pyrogens or other contaminants contraindicated for administration to an individual according to a selected route of administration.

In pharmaceutical dosage forms, the bifunctional degrader can be administered alone or in appropriate association, as well as in combination, with other pharmaceutically active compounds. The following methods and excipients are merely examples and are in no way limiting.

For oral preparations, the bifunctional degrader can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The bifunctional degraders can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The pharmaceutical composition may be in a liquid form, a lyophilized form or a liquid form reconstituted from a lyophilized form, where the lyophilized preparation is to be reconstituted with a sterile solution prior to administration. The standard procedure for reconstituting a lyophilized composition is to add back a volume of pure water (typically equivalent to the volume removed during lyophilization); however, solutions comprising antibacterial agents may be used for the production of pharmaceutical compositions for parenteral administration.

An aqueous formulation of the bifunctional degrader may be prepared in a pH buffered solution, e.g., at pH ranging from about 4.0 to about 8.0, such as from about 4.5 to about 7.5, e.g., from about 5.0 to about 7.0. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

### Method of use:

Disclosed herein are methods of using the bifunctional lysosomal targeting degraders of the present disclosure.

In certain aspects, provided herein are methods of degrading a membrane or extracellular protein. Such methods include contacting the membrane or extracellular protein with any of the bifunctional lysosomal targeting degraders of the present disclosure, under conditions in which the bifunctional lysosomal targeting degrader shuttles the membrane or extracellular protein to lysosome for degradation and the bifunctional lysosomal targeting degrader are recycled back out of cells and be catalytic. Such methods find use in a variety of applications. In certain aspects, the method is performed in vitro (e.g., in a tube, cell culture plate or well, or the like) and finds use, e.g., in testing and/or research applications. In other aspects, the method is performed in vivo (e.g., in an individual to whom the bifunctional degrader is administered) and finds use, e.g., in clinical/therapeutic applications.

In some embodiments, provided are methods that include administering to an individual in need thereof a therapeutically effective amount of any of the bifunctional degraders or any of the pharmaceutical compositions of the present disclosure. A variety of individuals are treatable according to the subject methods. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human.

In some embodiments, an effective amount of the bifunctional degrader (or pharmaceutical composition including same) is an amount that, when administered alone (e.g., in monotherapy) or in combination (e.g., in combination therapy) with one or more additional therapeutic agents, in one or more doses, is effective to reduce the symptoms of a medical condition of the individual (e.g., cancer) by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the bifunctional degrader or pharmaceutical composition.

In some embodiments, provided are methods that include administering to an individual having cancer a therapeutically effective amount of any of the bifunctional degraders or any of the pharmaceutical compositions of the present disclosure. According to such methods, the protein-binding moiety of the bifunctional degraders binds a membrane or extracellular protein that at least contributes to the individual's cancer, and where targeted degradation of the membrane or extracellular protein using the bifunctional degrader treats the individual's cancer. In certain aspects, the protein-binding moiety binds to a protein selected from a membrane receptor, a ligand for a membrane receptor, an immune inhibitory receptor, a ligand of an immune inhibitory receptor, an immune checkpoint molecule, an autoantibody, a secreted protein, and a mutated protein.

In certain embodiments, the individual has a cancer characterized by the presence of a solid tumor, a semi-solid tumor, a primary tumor, a metastatic tumor, or the like. In some embodiments, the individual has a cancer selected from breast cancer, melanoma, lung cancer, colorectal cancer, prostate cancer, glioma, bladder cancer, endometrial cancer, kidney cancer, leukemia (e.g., acute myeloid leukemia (AML)) liver cancer (e.g., hepatocellular carcinoma (HCC), such as primary or recurrent HCC), non-Hodgkin lymphoma, pancreatic cancer, thyroid cancer, any combinations thereof, and any sub-types thereof.

In any of the methods of using the bifunctional degraders of the present disclosure, in certain embodiments, the bifunctional degrader enhances degradation of the membrane or extracellular protein relative to degradation of the membrane or extracellular protein in the presence of the protein-binding moiety alone. Similarly, in any of the methods of using the bifunctional degraders of the present disclosure, according to some embodiments, the bifunctional degrader enhances degradation of the membrane or extracellular protein relative to degradation of the membrane or extracellular protein in the presence of the ligand of transferrin receptor or the protein-binding moiety alone.

By “treat”, “treating” or “treatment” is meant at least an amelioration of the symptoms associated with the medical condition (e.g., cell proliferative disorder, e.g., cancer) of the individual, where amelioration is used in a broad sense to refer to at least a reduction in the magnitude of a parameter, e.g., symptom, associated with the medical condition being treated. As such, treatment also includes situations where the medical condition, or at least symptoms associated therewith, are completely inhibited, e.g., prevented from happening, or stopped, e.g., terminated, such that the individual no longer suffers from the medical condition, or at least the symptoms that characterize the medical condition.

The bifunctional degrader or pharmaceutical composition may be administered to the individual using any available method and route suitable for drug delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, nasal, oral, and other enteral and parenteral routes of administration. In some embodiments, the administering is by parenteral administration. Routes of administration may be combined, if desired, or adjusted depending upon the bifunctional degrader and/or the desired effect. The bifunctional degraders or pharmaceutical compositions may be administered in a single dose or in multiple doses. In some embodiments, the bifunctional degrader or pharmaceutical composition is administered intravenously. In some embodiments, the bifunctional degrader or pharmaceutical composition is administered by injection, e.g., for systemic delivery (e.g., intravenous infusion) or to a local site.

### EXAMPLES

### Methods

Sources of reagents:

Biotin labeled human Transferrin (MilliporeSigma, a subsidiary of Merck KGaA, St. Louis, Missouri, USA, catalog no. T3915-5 MG).

Plasmids: Vigene Biosciences (Rockville, Maryland, USA).

Transferrin cDNA ORF Clone, Human, N-DYKDDDDK (SEQ ID NO:4) tag (FLAG®-brand), SinoBiological, Inc., Wayne, Pennsylvania, USA; catalog no. HG11019-NF).

Transferrin cDNA ORF Clone, Human, C-DYKDDDDK (SEQ ID NO:4) tag, SinoBiological; catalog no. HG11019-CF).

Mouse IgG uptake with transferrin (Tf)-biotin:

Cells were plated at 70% confluence in a 24-well plate. Media with or without serum supplemented with 50 nM of mouse anti-biotin-IgG-647 and 25 nM of biotin-labelled transferrin was sequentially added. The cells were incubated at 37° C. for indicated time points and then lysed for in gel fluorescence analysis.

Transferrin-biotin recycling assay:

Cells were plated at 70% confluence in a 12-well plate. Cells were starved by replacing complete growth media with the media without serum 1 h before treatment. Then cells were incubated with 50 nM of mouse anti-biotin-IgG-647 and 25 nM of biotin-labelled transferrin for 30 min, followed by 5-time washes with cold PBS. Cells were incubated with 2.5 μM Holo-TF and 100 μM deferoxamine mesylate for 30 or 90 min to prevent the re-entering of the Tf-biotin/target protein complex into the cells. Cell lysate and media were collected for in gel fluorescence analysis.

Comparison of the recycling of Tf-biotin, Fab-trimeric GalNAc (GN), and GN-biotin:

Huh7 cells were seeded at 70% confluence in a 12-well plate. Cells were starved by replacing complete growth media with the media without serum 1 h before treatment. Then cells were incubated with 50 nM of mouse anti-biotin-IgG-647 and 25 nM of biotin-labelled transferrin or 25 nM Fab-GN or 200 nM GN-biotin for 2 h, followed by 5-time washes with cold PBS. Cells were incubated with 2.5 μM Holo-TF and 100 μM deferoxamine mesylate or 2 μM GN-COOH for 2 h to prevent the re-entering of the Tf-biotin/target protein complex into the cells. Cell lysate and media were collected for in gel fluorescence analysis.

Mouse IgG uptake with Tf-Flag encoded by plasmids:

293 cells were transfected with plasmid expressing transferrin-Flag for 24 h. After washing and replacing with the media containing no serum, cells were treated with 25 nM anti-flag-647 for 24 or 36 h to allow the uptake of anti-flag-647. Cell lysates and media were collected for in gel fluorescence and western blot analysis.

Western blotting:

Cells were lysed in 1X RIPA lysis buffer containing 25 mM Tris, pH 7-8, 150 mM

NaCl, 0.1% (w/v) sodium dodecyl sulfate (SDS), 0.5% sodium deoxycholate, 1% (v/v) Triton X-100, protease inhibitor cocktail (Roche, one tablet per 10 mL) and 1 mM phenylmethylsulfonyl fluoride on ice for 10 min. The lysates were then centrifuged at 16000 g at 4° C. for 15 min and the supernatant was collected followed by measuring the protein concentration using BCA assay. Lysates were adjusted to the equal amount before mixed with the 4× Laemmli Loading Dye and heated at 99° C. for 5 min. After cooling down, samples were loaded onto 7.5% SDS-polyacrylamide gel electrophoresis and transferred to PVDF membrane. The membrane was first blocked in 5% (w/v) nonfat milk in the TBS-T washing buffer (137 mM NaCl, 20 mM Tris, 0.1% (v/v) Tween) and then incubated with primary antibodies at 4° C. overnight. After 3 washes with TBST, the membrane was incubated with secondary HRP-linked antibodies for 1 h, and then washed 3 times with TBST. Then the membrane was incubated in the Clarity ECL substrate for 3-5 min before acquiring the immunoblot using a “ChemiDoc”-brand MP Imaging System (BioRad Laboratories, Hercules, California, USA).

Biotin-labelled transferrin:

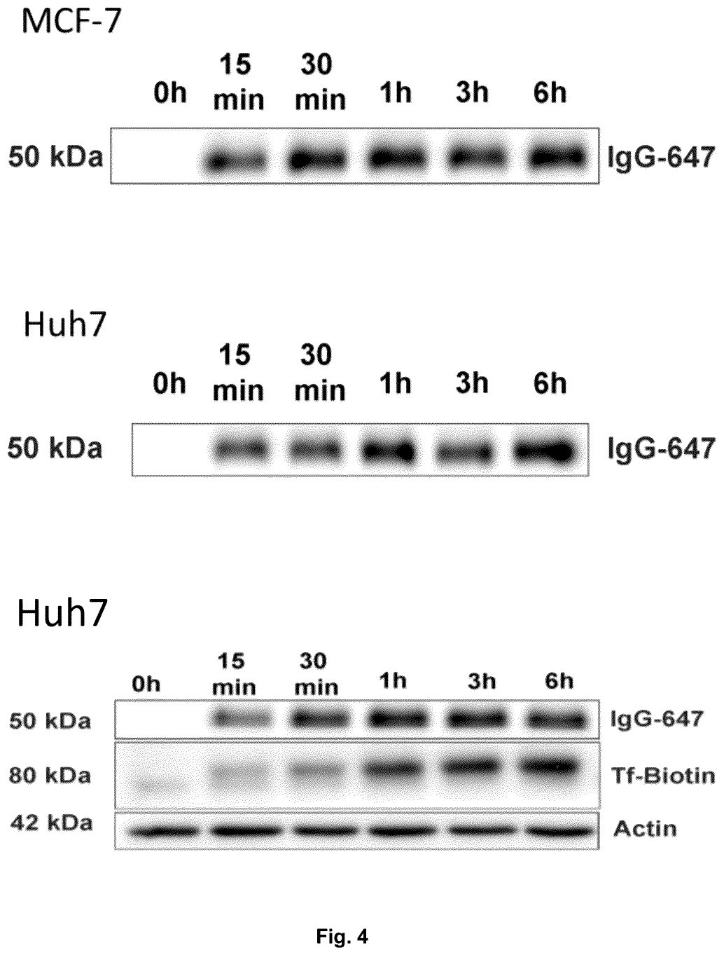

In this example, we used biotin-labelled transferrin (Tf-biotin) to evaluate the uptake of anti-biotin-647 (IgG-647), a fluorescent protein that can bind tightly to biotin. As shown in FIG. 4, significant uptake of IgG-647 was observed when the cells were treated with biotin-labelled Tf (25 nM of Tf-biotin and 50 nM of IgG-647 in MCF-7 and Huh7 cells).

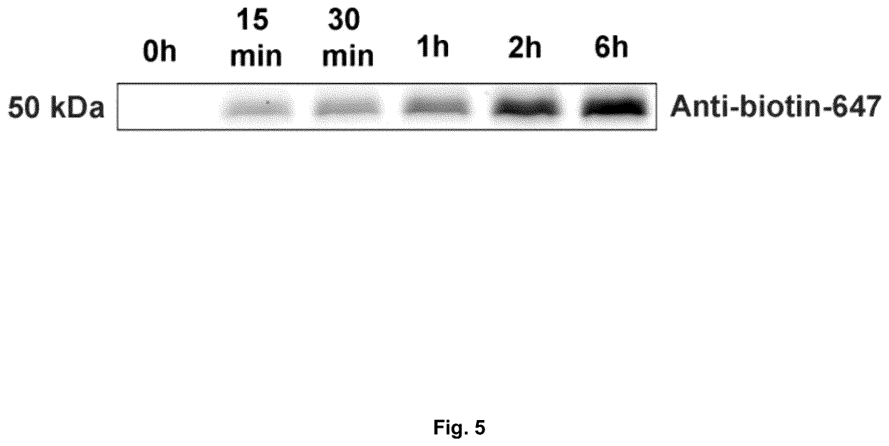

The above uptake experiment was done using regular media with serum. We subsequently realized that serum contains significant amount of Tf (˜25 μM). The uptake of anti-biotin-647 (IgG-647) in Huh7 cells in media without serum kept increasing until 6 h as shown in FIG. 5.

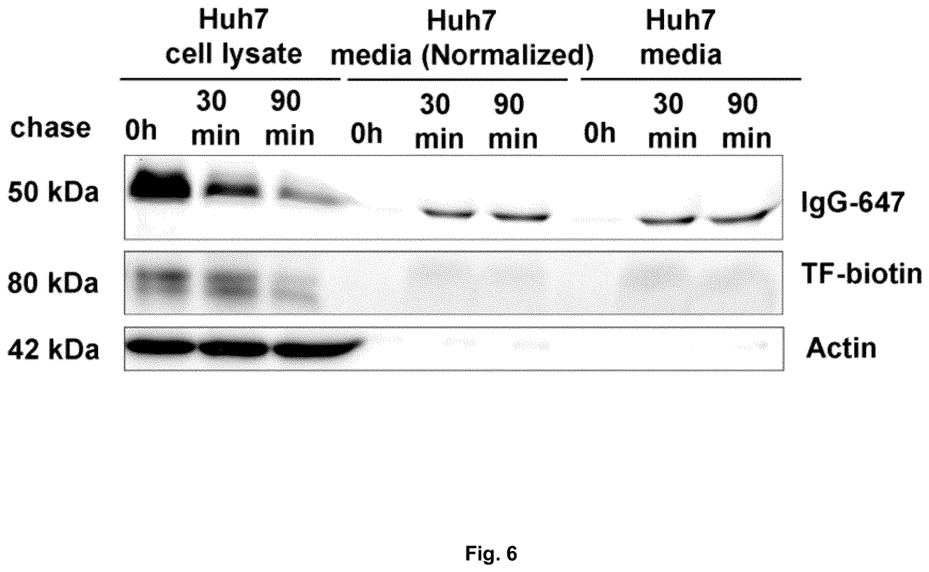

We then examined if the biotin-labelled Tf can travel through the membrane in and out as a catalytic lysosome targeting degrader by pause-chase experiments. The cells were incubated with Tf-biotin and anti-biotin-647 (IgG-647) for 30 min in media without serum to allow the cellular uptake of these two proteins through the action of Tf-receptor. The media was removed, and the cells were washed five times with cold PBS (pause). The level of IgG-647 and Tf-biotin inside the cells and in the media were examined at 0 h, 30 min, and 90 min as shown in FIG. 6. Our results showed that the amount of IgG-647 and Tf-biotin were decreased inside the cells and increased in the media during the chase phase, indicating that the biotinylated transferrin (Tf-biotin) can indeed travel through the membrane in and out together with the target protein anti-biotin-647.

During the chase phase, we also compared the amount of cellular IgG-647 with excess amount of holo-Tf and deferoxamine, which can essentially block the re-entering of Tf-biotin, and without these inhibitors. Holo-Tf competes with Tf-biotin for binding to Tf-receptor. Deferoxamine is an iron scavenger. Without iron, Tf stays at the apo-stage and does not bind to Tf-receptor. We found that there were more IgG-647 inside the cells without these inhibitors during the chase phase, suggesting that the Tf-biotin/IgG-647 can get into the cells before the pause, come out the cells during the chase phase, and get back into the cells during the chase phase.

We then did the same pause-chase experiment for tri-GalNAc-biotin and tri-GalNAc-antibody, our previously developed lysosome targeting degraders (Zhou et al., 2021, ACS Cent. Sci. 7:499-506). The amount of IgG-647 did not change in the media during the chase phase, indicating that tri-GalNAc-biotin and tri-GalNAc-antibody cannot come out from the cells together with the target protein anti-biotin-647 once they enter the cells.

For further applications, transferrin can be labeled with a small molecule ligand that can bind to a disease-causing target protein. The interaction between the small molecule ligand and the target protein will be much weaker than the interaction between biotin and its target anti-biotin antibody. The target protein will likely dissociate from the small molecule in endosome and less likely to come out of the cells together with the labelled transferrin.

Transfection with plasmid that expresses transferrin with a FLAG-tag:

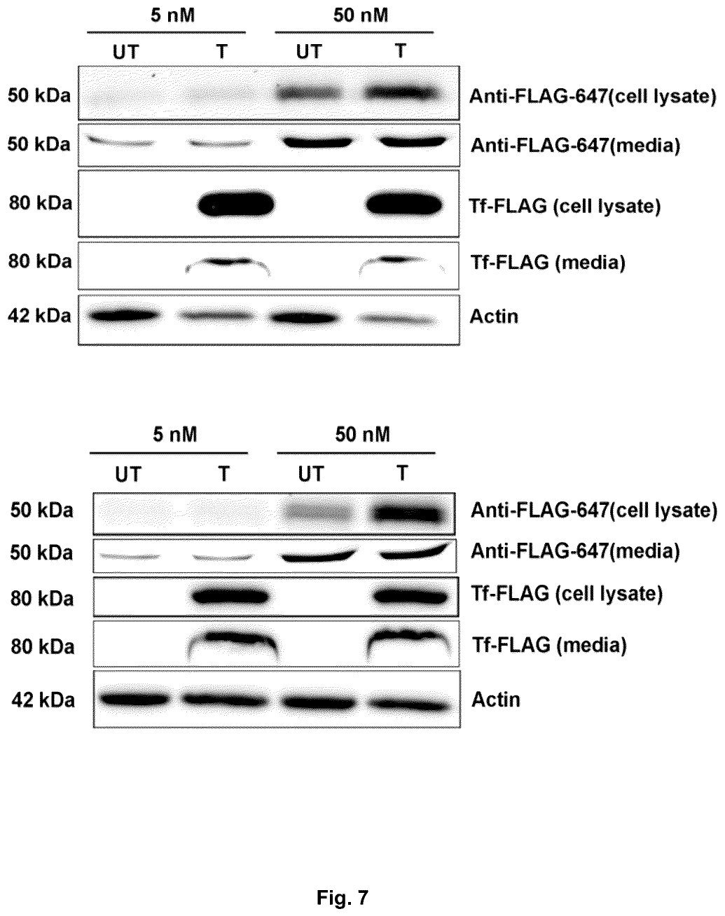

In this example, we did a transient transfection of plasmid (2.5 μg for 6×105 cells) that expresses Tf with a FLAG-tag on either the N-or C-termini. We found that the transfection efficiency of C-FLAG is much higher than N-FLAG in 293 cells. The plasmid for transferrin with a C-FLAG was then used for the uptake experiment. After 24 h of transfection, we then washed the media and added a fluorescent anti-FLAG-647 antibody (5 or 50 nM) to the media of the cell. We were able to observe significant amount of anti-FLAG-647 antibody in the cell lysate with transfected (T) cells, but not untransfected (UT) cells as shown in FIG. 7. This enables delivering mRNA of transferrin with a fused peptide that can bind disease-causing protein for targeted protein degradation. The resulting degrader should be catalytic.

Peptide binders of transferrin receptor:

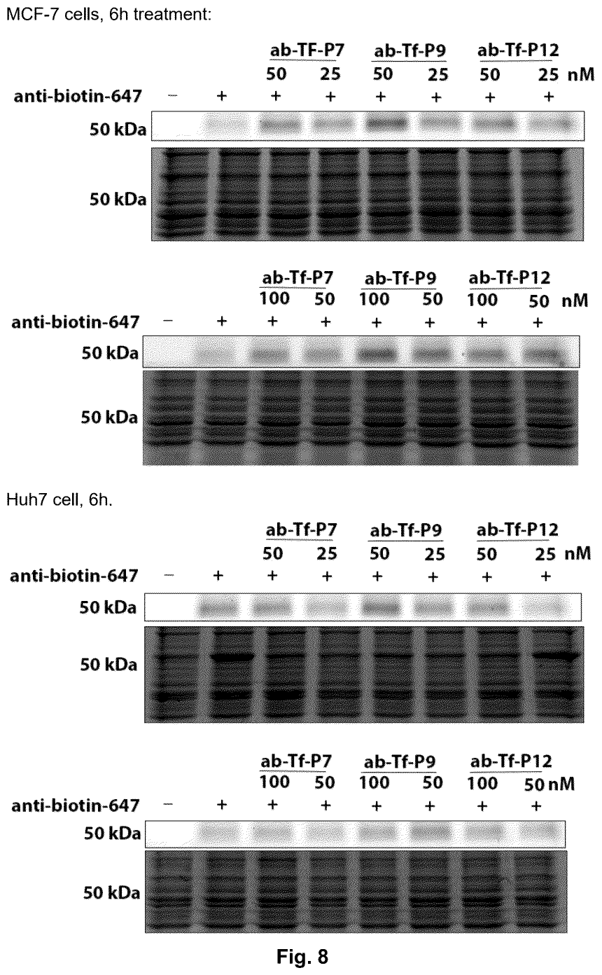

In this example, we attached each of the three peptides P7 (HAIYPRH; SEQ ID. NO: 1), P9 (GHKAKGPRK; SEQ ID NO:2) and P12 (THRPPMWSPVWP; SEQ ID NO:3) to an antibody that can bind to a fluorescent model target protein-anti-biotin-647. Three lysosome targeting degraders, ab-Tf-P7, ab-Tf-P9, and ab-Tf-P12 were prepared and tested for uptake of the model target protein. Degraders based on P9 peptide binder gives the best uptake (FIG. 8).

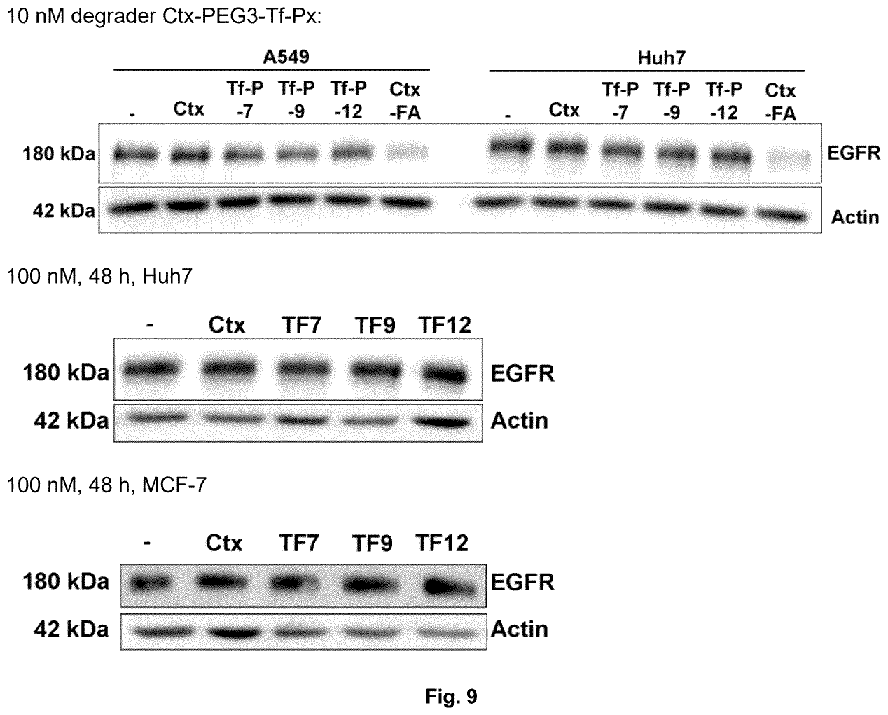

We also attached each of the P7, P9 and P12 peptides to Cetuximab (Ctx), antibody that can bind to a membrane target protein EGFR. (Ctx is available commercially from ImClone LLC, New York, New York, under the registered trademark “ERBITUX”®.) Three lysosome targeting degraders, Tf-P7, Tf-P9, and Tf-P12 were prepared with PEG3 as the linker and tested for the degradation of EGFR. Ctx-labeled folate (Ctx-FA) was used as a positive control. As shown in FIG. 9, the degraders with a short linker, PEG3, did not show obvious degradation of EGFR, though Tf-P7 appears to be better than others.

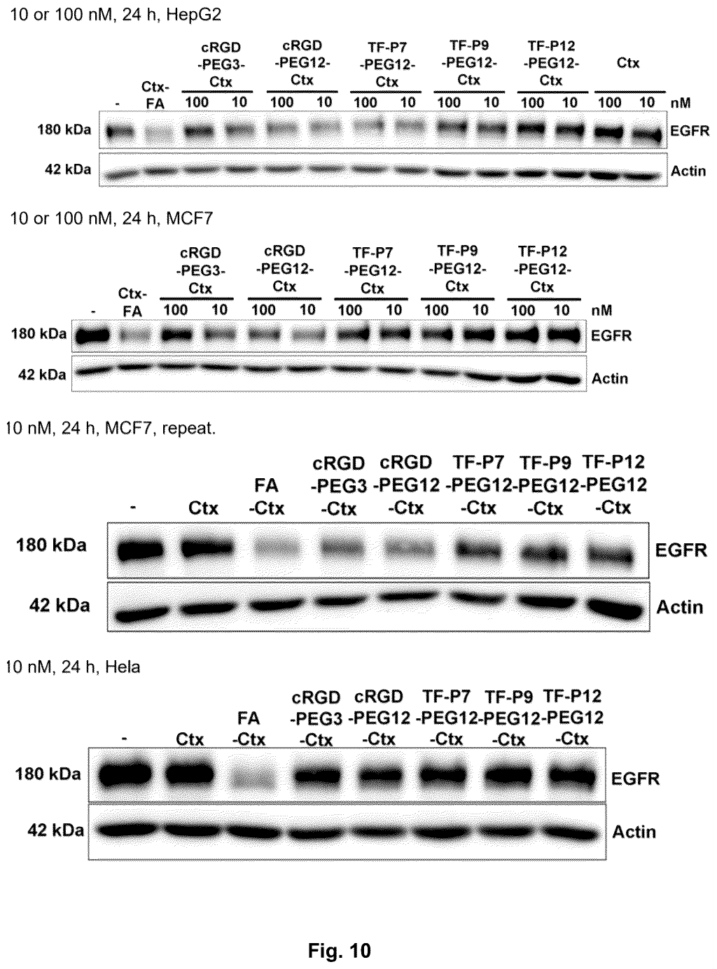

We then used a longer linker, PEG12, between the peptide binder of transferrin receptor and Ctx. Ctx-FA was used as a positive control. The results indicate that degraders based on P7 peptide binder of transferrin receptor show higher degradation efficiency than the other two in some cancer cells. (FIG. 10).

The results show that peptide binders of transferrin receptor can be used for targeted protein degradation. For the construction of bifunctional lysosome targeting degraders, it is relatively straightforward to attach peptide ligands to antibodies. These peptide binders can complement transferrin for the development of lysosome targeting degraders that recruit transferrin receptors.

What is claimed is:

1. A bifunctional lysosomal targeting degrader, comprising:
a ligand configured to bind to transferrin receptor as a shuttle molecule for lysosome degradation; operationally linked to
a protein-binding moiety configured to bind a membrane or extracellular protein.

2. The bifunctional lysosomal targeting degrader of claim 1, wherein the ligand is transferrin.

3. The bifunctional lysosomal targeting degrader of claim 1, wherein the ligand is an antibody that binds to transferrin receptor.

4. The bifunctional lysosomal targeting degrader of claim 1, wherein the protein-binding moiety binds a membrane protein.

5. The bifunctional lysosomal targeting degrader of claim 4, wherein the peptide binder of transferrin receptor is selected from the group consisting of sequences HAIYPRH (SEQ ID NO:1), GHKAKGPRK (SEQ ID NO:2), and THRPPMWSPVWP (SEQ ID NO:3).

6. The bifunctional lysosomal targeting degrader of claim 5, wherein the peptide binder of transferrin receptor has a sequence of HAIYPRH (SEQ ID NO:1).

7. The bifunctional lysosomal targeting degrader of claim 1, wherein the protein-binding moiety binds an extracellular protein.

8. The bifunctional lysosomal targeting degrader of claim 1, wherein the protein-binding moiety is selected from a polypeptide, a ligand, an aptamer, a nanoparticle, and a small molecule.

9. The bifunctional lysosomal targeting degrader of claim 8, wherein the bifunctional degrader is a fusion protein comprising the ligand fused to the protein-binding moiety.

10. The bifunctional lysosomal targeting degrader of claim 9, wherein the bifunctional degrader further comprises a spacer between the ligand and the protein-binding moiety.

11. The bifunctional lysosomal targeting degrader of claim 9, wherein the bifunctional degrader is expressed in cells by introducing to the cells an expression vector that contains nucleic acids encoding the bifunctional degrader.

12. The bifunctional lysosomal targeting degrader of claim 1, wherein the protein-binding moiety is a small molecule.

13. The bifunctional lysosomal targeting degrader of claim 1, wherein the protein-binding moiety is a polypeptide.

14. The bifunctional lysosomal targeting degrader of claim 1, wherein the protein-binding moiety is an antibody.

15. The bifunctional lysosomal targeting degrader of claim 1, further comprising one or more linkers configured to facilitate conjugation of the ligand to the protein-binding moiety.

16. The bifunctional lysosomal targeting degrader of claim 1, wherein the bifunctional lysosomal targeting degrader selectively targets cells that express transferrin receptors.

17. The bifunctional lysosomal targeting degrader of claim 16, wherein the cells that express transferrin receptors are cancer cells.

18. A pharmaceutical composition comprising the bifunctional lysosomal targeting degrader of claim 1.

19. The pharmaceutical composition of claim 18, further comprising a pharmaceutically acceptable carrier.

20. A method of degrading a membrane or extracellular protein, comprising:
contacting the membrane or extracellular protein with the bifunctional lysosomal targeting degrader of claim 1;
wherein the bifunctional lysosomal targeting degrader shuttles the membrane or extracellular protein to lysosome for degradation.

21. A method comprising administering to an individual in need thereof a therapeutically effective amount of the pharmaceutical composition of claim 18.

22. The method of claim 21, wherein the individual is a human.

23. The method of claim 21, wherein the individual has cancer.


SEQUENCE LISTING (4 sequences)

SEQ ID NO: 1 - protein, 7 aa, synthetic construct
HAIYPRH

SEQ ID NO: 2 - protein, 9 aa, synthetic construct
GHKAKGPRK

SEQ ID NO: 3 - protein, 12 aa, synthetic construct
THRPPMWSPVWP

SEQ ID NO: 4 - protein, 8 aa, Homo sapiens
DYKDDDDK